(19) United States
(12) Patent Application Publication
(10) Pub. No.: US 2024/0034988 A1
(43) Pub. Date: Feb. 1, 2024

(54) ADVANCED MANUFACTURED TRANSWELL USING SYNTHETIC BIOINK FOR CELL CULTURE AND IN VITRO TISSUE MODELS

(71) Applicant: Lung Biotechnology PBC, Silver Spring, MD (US)

(72) Inventors: Victor Hernandez-Gordillo, Cambridge, MA (US); Anisha Beladia, Silver Spring, MD (US); Roya Samanipour, Manchester, NH (US); Abdulrahman Alsasa, Silver Spring, MD (US); Katherine Russo, Merrimack, NH (US); Ziwen Wang, Silver Spring, MD (US); Gregory Hurst, Bedford, NH (US); Barbara Nsiah, Manchester, NH (US); Luis Alvarez, Lexington, MA (US)

(73) Assignee: Lung Biotechnology PBC, Silver Spring, MD (US)

(21) Appl. No.: 18/227,001

(22) Filed: Jul. 27, 2023

Related U.S. Application Data
(60) Provisional application No. 63/392,724, filed on Jul. 27, 2022.

(51) Int. Cl. C12N 5/00; C12M 1/12; B33Y 80/00

(52) U.S. Cl. CPC C12N 5/0062; C12M 25/04; B33Y 80/00; C12N 2513/00

(57) ABSTRACT

An advanced manufactured transwell (AM-transwell), the AM-transwell comprises: a lower chamber; an upper chamber; a membrane disposed between the lower chamber and the upper chamber; and one or more legs. The one or more legs form at least a portion of the lower chamber. One or more of the lower chambers, the upper chamber, the membrane and the one or more legs is printed using a synthetic bioink. Methods for making and using the AM-transwell are also disclosed.

### CROSS-REFERENCE TO RELATED APPLICATIONS

This application claims priority to U.S. Provisional Application No. 63/392,724, filed Jul. 27, 2022, which is incorporated by reference in its entirety.

### SEQUENCE LISTING

The instant application contains a Sequence Listing which has been submitted in XML, format via Patent Center and is hereby incorporated by reference in its entirety. Said XML copy, created on Oct. 9, 2023, is named 080618-2260_SL.xml and is 6,334 bytes.

### TECHNICAL FIELD

The present application relates to 3D printed structures.

### BACKGROUND

In vitro assessment of epethileial and endothelial barrier function is traditionally achieved using commercially available plastic transwells (e.g., Corning) that contain a porous membrane made of a polyester (PET) or polycarbonate (PC). These membranes are “stiff” and do not allow for modification of their mechanical properties.

### SUMMARY

The systems and methods of the present disclosure include 3D printed advanced manufactured (AM) transwells using synthetic bioinks. The 3D printed AM-transwell contains a printable membrane that is tunable in thickness, porosity, and mechanical properties. The 3D printed AM-transwells may be surface modified to allow epithelial and endothelial cells barrier formation, followed by barrier function assessment.

In an embodiment, an advanced manufactured transwell (AM-transwell), the AM-transwell comprises: a) a lower chamber; b) an upper chamber; c) a membrane disposed between the lower chamber and the upper chamber; and d) one or more legs.

In an embodiment, the one or more legs form at least a portion of the lower chamber.

In an embodiment, the AM-transwell is a cylinder, a cube, a cuboid, a truncated cone, a truncated pyramid, a truncated sphere or a combination thereof. In an embodiment, the AM-transwell is a cube. In an embodiment, the AM-transwell is a cylinder.

In an embodiment, the AM-transwell has a height of about 10 mm to about 16 mm, and any range or value there between, and a thickness from about 1 mm to about 3 mm, and any range or value there between. In an embodiment, the AM-transwell has a height of about 13 mm and a thickness of about 2 mm.

In an embodiment, the lower chamber has a height from about 2 mm to about 4 mm, and any range or value there between, and the upper chamber has a height from about 8 to about 12 mm, and any range or value there between. In an embodiment, the lower chamber has a height of about 3 mm and the upper chamber has a height of about 10 mm.

In an embodiment, the membrane has a thickness from about 1 mm to about 3 mm, and any range or value there between. In an embodiment, the membrane has a thickness of about 2 mm.

In an embodiment, one or more of the lower chambers, the upper chamber, the membrane and the one or more legs is printed using a synthetic bioink.

In an embodiment, the synthetic bioink comprises one or more of a degradable peptide ink and triacrylate peptide ink.

In an embodiment, the synthetic bioink comprises one or more of: HPA, in an amount from about 3% to about 10%, and any range or value there between; PEGDA3400, in an amount from about 5% to about 20%, and any range or value there between; PEGDA6000, in an amount from about 5% to about 20%, and any range or value there between; PEGDA575, in an amount from about 1% to about 20%, and any range or value there between; PEGDA700, in an amount from about 1% to about 20%, and any range or value there between; PEGTAC, in an amount from about 1% to about 5% and any range or value there between; PEO, in an amount from about 0.1% to about 5%, and any range or value there between; NAP, in an amount from about 1% to about 3%, and any range or value there between; LAP, in an amount from about 1% to about 3%, and any range or value there between; and UV386A (386 nm visible dye), in an amount from about 0.1% to about 0.5%, and any range or value there between.

In an embodiment, the synthetic bioink further comprises: a water balance.

In an embodiment, the synthetic bioink further comprises: a buffer solution comprising 0.1 M HEPES in water and 1X PBS at pH of 7.2.

In an embodiment, the synthetic bioink further comprises: mono-cysteine peptide, in an amount from 0.5 mM to 20 mM, and any range or value there between; and dicysteine peptide, in an amount from 0.5 mM to 20 mM, and any range or value there between.

In an embodiment, the dicysteine peptide is Matrix Metalloproteinase (MMP) degradable.

In an embodiment, the mono-cysteine peptide comprises one or more of RGDS (SEQ ID NO: 1), PHSRNKRGDS (SEQ ID NO: 2), IKVAV (SEQ ID NO: 3), AG73 (SEQ ID NO: 5), GFOGER (SEQ ID NO: 4), Bm-Binder, and Fn-Binder.

In an embodiment, a method of making an advanced manufactured transwell (AM-transwell) comprises: a) printing one or more of a lower chamber, an upper chamber and a membrane of the AM-transwell using a 3D printing technique; and b) assembling and/or printing the AM-transwell as described herein to form assembled AM-transwells.

In an embodiment, step b) prints at least 3 assembled AM-transwells and any range or value therein. In an embodiment, step b) prints at least 20 assembled AM-transwells. In an embodiment, step b) prints at least 50 assembled AM-transwells.

In an embodiment, the 3D printing technique is one or more of digital light projection (DLP) printing technique, sterolithography (SLA) printing technique, extrusion 3D printing technique or selective laser sintering 3D printing technique or a combination thereof. In an embodiment, the 3D printing technique is a digital light projection (DLP) printing technique.

In an embodiment, the method further comprises: c) optionally, storing the assembled AM-transwells at 4° C. until needed.

In an embodiment, the method further comprises: c) transferring each of the AM-transwells into 1X DPBS Ca+/Mn+ in tubes; d) decanting the 1X DPBS Ca+/Mn+ from the tubes and washing each the AM-transwells two additional times in DPBS Ca+/Mn+ for about 5 minutes to form first washed AM-transwells; and 3) transferring the first washed AM-transwells to sterile tubes. In an embodiment, the method further comprises: c) transferring each of the AM-transwells into about 35 mL DPBS Ca+/Mn+ in about 50 mL tubes; d) decanting the DPBS Ca+/Mn+ from the tubes and washing each the AM-transwells two additional times in about 35 mL DPBS Ca+/Mn+ for about 5 minutes to form first washed AM-transwells; and e) transferring the first washed AM-transwells to sterile 50 mL tubes. In an embodiment, steps c) and d) volume of DPBS Ca+/Mn+ and size of tube depends on the number of AM-transwells being transferred. The AM-transwells just need to be enclosed in some buffered deionized water solution.

In an embodiment, the method further comprises: f) incubating the first washed AM-transwells in 1X DPBS Ca−/Mn− supplemented with antibiotic/antimycotic (anti-anti) overnight to form sterilized AM-transwells.

In an embodiment, the method further comprises: g) decanting the 1X DPBS Ca−/Mn− anti-anti and washing the incubated AM-transwells in about 35 mL of 1X PBS/0.1 M HEPES anti-anti buffer solution for about 4 hours to form second washed AM-transwells; and h) decanting the 1X PBS/0.1 M HEPES anti-anti buffer solution from the tubes and incubating the second washed AM-transwells in about 35 mL cell culture media in tubes for about 2 days or over a weekend. In an embodiment, the method further comprises: g) decanting the 1X DPBS Ca−/Mn− anti-anti and washing the incubated AM-transwells in about 35 mL 1X PBS/0.1 M HEPES anti-anti buffer solution for about 4 hours to form second washed AM-transwells; and h) decanting the 1X PBS/0.1 M HEPES anti-anti buffer solution from the tubes and incubating the second washed AM-transwells in about 35 mL cell culture media in about 50 mL tubes for about 2 days or over a weekend.

In an embodiment, a method of using an advanced manufactured transwell (AM-transwell) comprises: a) cell-seeding at least one side of the membrane of the AM-transwell as described herein for in vitro cells studies.

In an embodiment, a method of using an advanced manufactured transwell (AM-transwell) comprises: a) cell-seeding both sides of the membrane of the AM-transwell as described herein for in vitro cells studies.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

The details of one or more implementations of the subject matter described in this specification are set forth in the accompanying drawings and the description below. Other features, aspects, and advantages of the subject matter will become apparent from the description, the drawings, and the claims.

FIG. 1 illustrates a commercially-available plastic transwell (prior art);

FIG. 2A shows a upper perspective view of drawing of an AM-transwell according to an embodiment of the present invention;

FIG. 2B shows a lower perspective view of a drawing of the AM-transwell of FIG. 2A;

FIG. 3A shows a top view of a drawing of an AM-transwell according to an embodiment of the present invention;

FIG. 3B shows a side view of a drawing of the AM-transwell according to FIG. 3A;

FIG. 4 depicts a photograph of a 3D printed AM-transwell using a synthetic bioink and a digital light projection (DLP) printer;

FIG. 5 depicts a photograph of a 3D printed membrane using a synthetic bioink and a digital light projection (DLP) printer;

FIG. 6 shows a photograph of exemplary 3D printed AM-transwell having a cylindrical shape using a synthetic bioink and a LS80 printer;

FIG. 7 depicts a photograph of an exemplary 3D printed AM-transwell having a cube shape using a synthetic bioink and a BLF20 printer;

FIG. 8 depicts a photograph of an exemplary 3D printed AM-transwell having a cylindrical shape using a synthetic bioink and a BLF20 printer.

FIG. 9 shows a schematic of an experimental set-up for a permeability assay of AM-transwells;

FIG. 10 shows a schematic of an experimental set-up for a serial dilution for the permeability assay of AM-transwells;

FIG. 11 shows a standard curve chart of Concentration (μg/mL) vs. Relative Fluorescence Units for the permeability assay of AM-transwells (Bionks AG71 through AG76);

FIG. 12 shows a chart of Time vs. FITC-Dextran Concentration (μg/mL) for the permeability assay of AM-transwells (Bionks AG71 through AG76);

FIG. 13A shows a chart of Time vs. FITC-Dextran Concentration (μg/mL) for the permeability assay of AM-transwells (Bionk AG71);

FIG. 13B shows a chart of Time vs. FITC-Dextran Concentration (μg/mL) for the permeability assay of an AM-transwell (Bionk AG72);

FIG. 13C shows a chart of Time vs. FITC-Dextran Concentration (μg/mL) for the permeability assay of an AM-transwell (Bionk AG73);

FIG. 13D shows a chart of Time vs. FITC-Dextran Concentration (μg/mL) for the permeability assay of an AM-transwell (Bionk AG74);

FIG. 13E shows a chart of Time vs. FITC-Dextan Concentration (μg/mL) for the permeability assay of an AM-transwell (Bionk AG75);

FIG. 13F shows a chart of Time vs. FITC-Dextran Concentration (μg/mL) for the permeability assay of an AM-transwell (Bionk AG76);

FIG. 14 shows a chart of Concentration (μg/mL) vs. Relative Fluorescence Units for the permeability assay of an AM-transwell (Bionks AG77 through AG80);

FIG. 15 shows a chart of Time vs. Percentage FITC-Dextran in the lower chamber compartment for the permeability assay of AM-transwells (Bionks AG77 through AG80);

FIG. 16A shows a chart of Time vs. Percentage FITC-Dextran in the lower chamber compartment for the permeability assay of an AM-transwell (Bionk AG77);

FIG. 16B shows a chart of Time vs. Percentage FITC-Dextran in the lower chamber compartment for the permeability assay of an AM-transwell (Bionk AG78);

FIG. 16C shows a chart of Time vs. Percentage FITC-Dextran in the lower chamber compartment for the permeability assay of an AM-transwell (AG79);

FIG. 16D shows a chart of Time vs. Percentage FITC-Dextran in the lower chamber compartment for the permeability assay of an AM-transwell (AG80); and

FIG. 17 shows a chart of Compartment vs. Percent Media Recovered from an AM-transwell after three days.

### DETAILED DESCRIPTION

The following detailed description of various embodiments of the present invention references the accompanying drawings, which illustrate specific embodiments in which the invention can be practiced. While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains. Therefore, the scope of the present invention is defined only by the appended claims, along with the full scope of equivalents to which such claims are entitled.

### Commercial Transwell

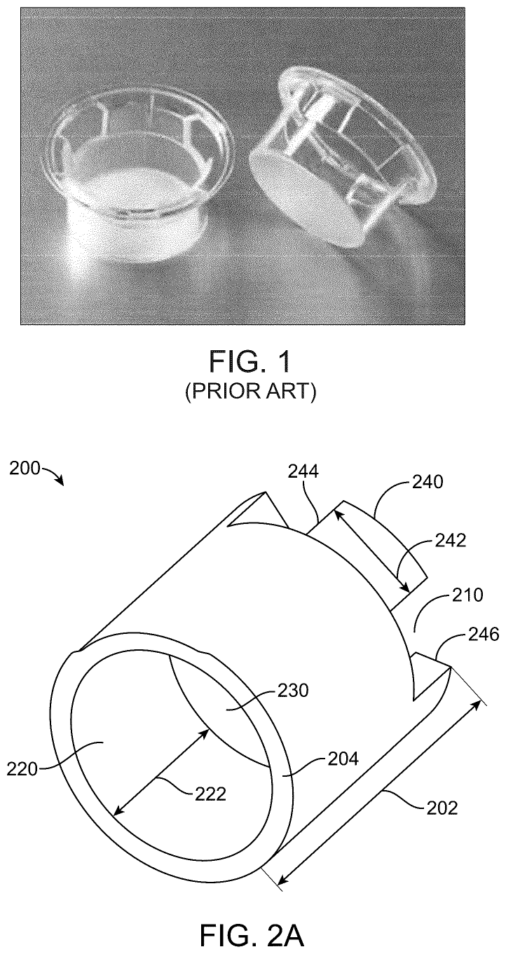

FIG. 1 illustrates a commercially available plastic transwell 100 (prior art).

### 3D Printed Transwell

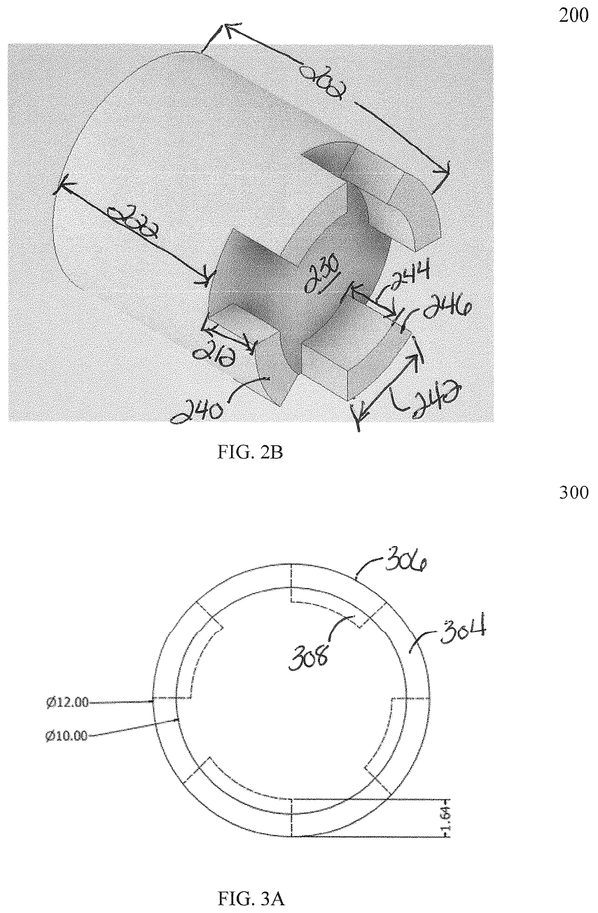

FIG. 2A shows an upper perspective view of drawing of an AM-transwell 200 according to an embodiment of the present invention; and FIG. 2B shows a lower perspective view of a drawing of the AM-transwell 200 of FIG. 2A.

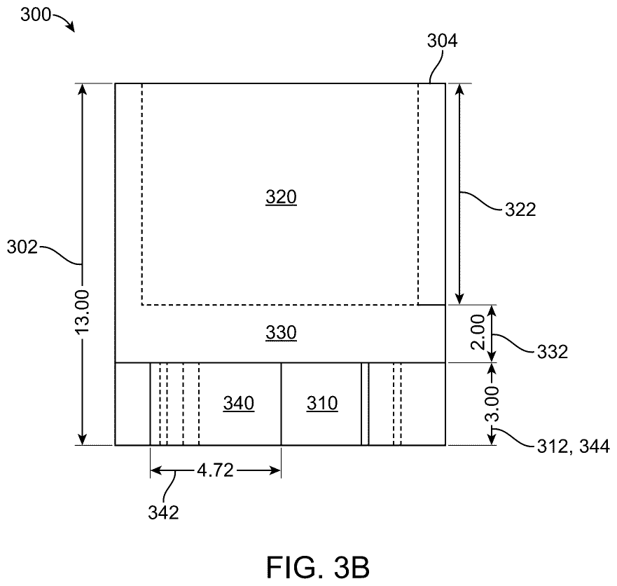

FIG. 3A shows a top view of a drawing of an AM-transwell 300 according to an embodiment of the present invention; and FIG. 3B shows a side view of a drawing of the AM-transwell 300 according to FIG. 3A.

As shown in FIGS. 2A-2B and 3A-3B, the AM-transwell 200, 300 has a lower chamber 210, 310, an upper chamber 220, 320, a membrane 230, 330 disposed between the lower chamber 210, 310 and the upper chamber 220, 320, and one or more legs 240, 340.

In an embodiment, the one or more legs 240, 340 form at least a portion of the lower chamber 210, 310.

In an embodiment, the AM-transwell is a cylinder, a cube, a cuboid, a truncated cone, a truncated pyramid, a truncated sphere or a combination thereof. In an embodiment, the AM-transwell is a cube. See e.g., FIG. 7. In an embodiment, the AM-transwell 200, 300 is a cylinder. See e.g., FIGS. 2A-2B & 3A-3B.

In an embodiment, the AM-transwell 200, 300 has a height 202, 302 of about 10 mm to about 16 mm and any range or value there between, and a thickness 204, 304 from about 1 mm to about 3 mm, and any range or value there between. In an embodiment, the AM-transwell 200, 300 has a height 202, 302 of about 13 mm and a thickness 204, 304 of about 2 mm.

In an embodiment, the thickness 202, 302 of the AM-transwell 200, 300 depends on an interface 308 of a mini-lobe 306.

In an embodiment, the lower chamber 210, 310 has a height 212, 312 from about 2 mm to about 4 mm, and any range or value there between, and the upper chamber 220, 320 has a height 222, 322 from about 8 to about 12 mm, and any range or value there between. In an embodiment, the lower chamber 210, 310 has a height 212, 312 of about 3 mm and the upper chamber 220, 320 has a height 222, 322 of about 10 mm.

In an embodiment, the membrane 230, 330 has a thickness 332 from about 1 mm to about 3 mm, and any range or value there between. In an embodiment, the membrane 230, 330 has a thickness 332 of about 2 mm.

In an embodiment, an inner well of the upper chamber 220, 320 supports lung epithelial cells (SAEC, Small airway epithelial cells) growth on inside.

In an embodiment, the one or more legs 240, 340 have a length 242, 342 from about 3.7 mm to about 5.7 mm, and any range or value there between. In an embodiment, the one or more legs 240, 340 have a length 242, 342 of about 4.72 mm.

In an embodiment, the one or more legs 240, 340 have a height 244, 344 from about 2 mm to about 4 mm, and any range or value there between. In an embodiment, the one or more legs 240, 340 have a height 244, 344 of about 3 mm.

In an embodiment, the one or more legs 240, 340 have a width 246 from about 0.6 mm to about 2.6 mm, and any range or value there between. In an embodiment, the one or more legs 240, 340 have a width 246 of about 1.64 mm.

In an embodiment, the one or more legs 240, 340 support lung endothelial cells (PAEC, Pulmonary airway endothelial cells) growth on the bottom.

In an embodiment, one or more of the lower chambers, the upper chambers, the membrane and the one or more legs is printed using a synthetic bioink.

In an embodiment, the synthetic bioink comprises one or more of a degradable ink and triacrylate peptide ink.

In an embodiment, the synthetic bioink comprises one or more of: HPA, in an amount from about 3% to about 10%, and any range or value there between; PEGDA3400, in an amount from about 5% to about 20%, and any range or value there between; PEGDA6000, in an amount from about 5% to about 20%, and any range or value there between; PEGDA575, in an amount from about 1% to about 20%, and any range or value there between; PEGDA700, in an amount from about 1% to about 20% and any range or value there between; PEGTAC, in an amount from about 1% to about 5%, and any range or value there between; PEO, in an amount from about 0.1% to about 5%, and any range or value there between; NAP, in an amount from about 1% to about 3%, and any range or value there between; LAP, in an amount from about 1% to about 3%, and any range or value there between; and UV386 A, in an amount from about 0.1% to about 0.5%, and any range or value there between.

In an embodiment, the synthetic bioink further comprises: Water, in an amount as a balance.

In an embodiment, the synthetic bioink further comprises: a buffer solution comprising 0.1 M HEPES in water and 1X PBS at pH of 7.2.

In an embodiment, the synthetic bioink further comprises: mono-cysteine peptide, in an amount from 0.5 mM to 20 mM, and any range or value there between; and dicysteine peptide, in an amount from 0.5 mM to 20 mM, and any range or value there between.

In an embodiment, the dicysteine peptide is MMP degradable.

In an embodiment, the mono-cysteine peptide comprises one or more of RGDS (SEQ ID NO: 1 ), PHSRNKRGDS (SEQ ID NO: 2 ), IKVAV (SEQ ID NO: 3 ), GFOGER (SEQ ID NO: 4 ), and ECM-binders (BM-binder, FN-Binder).

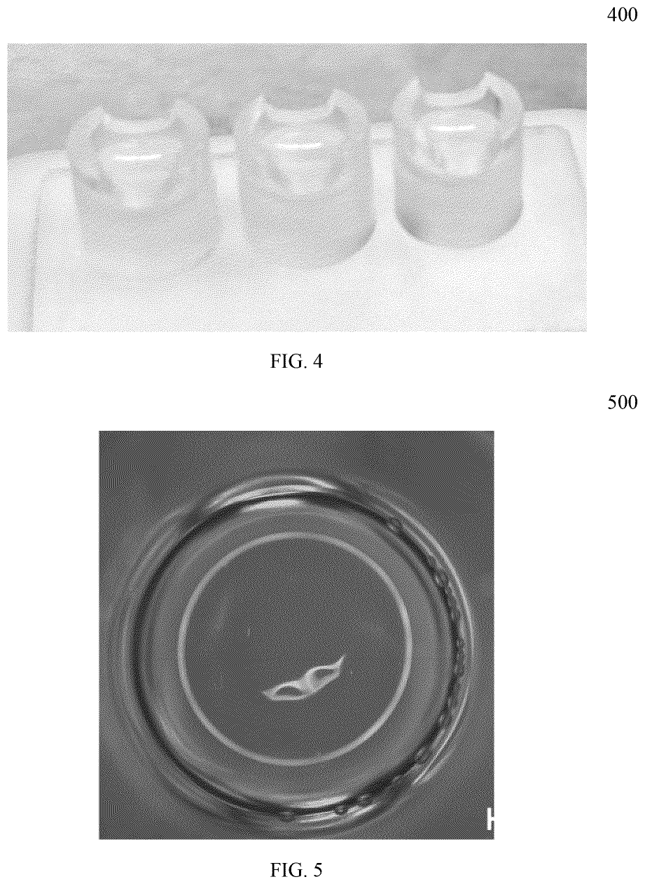

FIG. 4 depicts a photograph of a 3D printed AM-transwell using a synthetic bioink and a digital light projection (DLP) printer; and FIG. 5 depicts a photograph of a 3D printed membrane using a synthetic bioink and a digital light projection (DLP) printer.

### Exemplary Bioink Ink Formulations and Print Settings for LS80 Printer

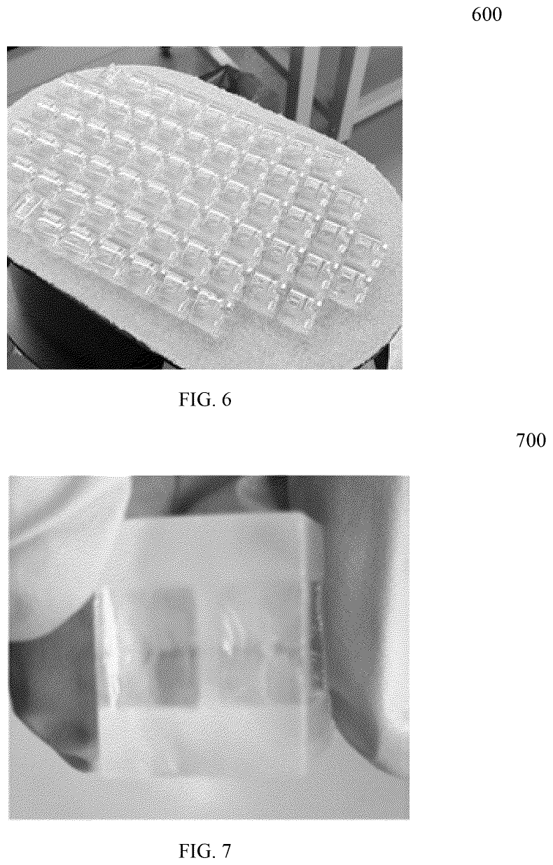

FIG. 6 shows a photograph of exemplary 3D printed AM-transwells having a cylindrical shape using a synthetic bioink and a LS80 printer.

### Example 1 (AG71)

| | | Actual | |
|---|---|---|---|
| | Registry | Concentration | Units |
| PEGDA 700 | PEGDA 700 | 2.85-3.15 | % |
| PEGDA 6000 | 40% PEGDA 6000 | 4.75-5.25 | % |
| HEPES | 1M HEPES pH 7.3 | 0.095-0.105 | M |
| 10X PBS | DPBS 10X Ca, Mg −/− | 0.95-1.05 | X |
| MMP | MMP - Crosslinker | 9.5-10.5 | mM |
| PHSRNK-RGDS | PHSRNKRGDS | 1.9-2.1 | mM |
| HPA | Hydroxylpropyl | 9.5-10.5 | % |
| | Acrylate (HPA) | | |
| NAP | NAP | 1.425-1.575 | % |
| UV386 | UV 386A | 0.114-0.126 | % |
| Water | | Balance | |

### Example 2 (AG72)

| | | Actual | |
|---|---|---|---|
| | Registry | Concentration | Units |
| PEG TAC | PEGTAC | 2.85-3.15 | % |
| PEGDA 6000 | 40% PEGDA 6000 | 4.75-5.25 | % |
| HEPES | 1M HEPES pH 7.3 | 0.095-0.105 | M |
| 10X PBS | DPBS 10X Ca, Mg −/− | 0.95-1.05 | X |
| HPA | Hydroxylpropyl | 9.5-10.5 | % |
| | Acrylate (HPA) | | |
| NAP | NAP | 1.425-1.575 | % |
| UV386 | UV 386A | 0.171-0.189 | % |
| Water | | Balance | |

### Example 3 (AG73)

| | | Actual | |
|---|---|---|---|
| | Registry | Concentration | Units |
| PEG TAC | PEGTAC | 2.85-3.15 | % |
| PEGDA 6000 | 40% PEGDA 6000 | 4.75-5.25 | % |
| HEPES | 1M HEPES pH 7.3 | 0.09500.105 | M |
| 10X PBS | DPBS 10X Ca, Mg —/— | 0.95-1.05 | X |
| MMP | MMP—Crosslinker | 9.5-10.5 | mM |
| PHSRNK-RGDS | PHSRNKRGDS | 1.9-2.1 | mM |
| HPA | Hydroxylpropyl Acrylate | 9.5-10.5 | % |
| | (HPA) | | |
| NAP | NAP | 1.425-1.575 | % |
| UV386 | UV 386A | 0.171-0.189 | % |
| Water | | Balance | |

### Example 4 (AG74)

| | | Actual | |
|---|---|---|---|
| | Registry | Concentration | Units |
| PEGDA700 | PEGDA700 | 3.325-3.675 | % |
| PEGDA 6000 | 40% PEGDA 6000 | 4.75-5.25 | % |
| HEPES | 1M HEPES pH 7.3 | 0.095-0.105 | M |
| 10X PBS | DPBS 10X Ca, Mg —/— | 0.95-1.05 | X |
| HPA | Hydroxylpropyl Acrylate | 9.5-10.5 | % |
| | (HPA) | | |
| NAP | NAP | 1.425-1.575 | % |
| UV386 | UV 386A | 0.171-0.189 | % |
| Water | | Balance | |

### Example 5 (AG75)

| | | Actual | |
|---|---|---|---|
| | Registry | Concentration | Units |
| PEGDA700 | PEGDA700 | 3.325-3.675 | % |
| PEGDA 6000 | 40% PEGDA 6000 | 4.75-5.25 | % |
| HEPES | 1M HEPES pH 7.3 | 0.095-0.105 | M |
| 10X PBS | DPBS 10X Ca, Mg —/— | 0.95-1.05 | X |
| MMP | MMP—Crosslinker | 9.5-10.5 | mM |
| PHSRNK-RGDS | PHSRNKRGDS | 1.9-2.1 | mM |
| HPA | Hydroxylpropyl Acrylate | 9.5-10.5 | % |
| | (HPA) | | |
| NAP | NAP | 1.425-1.575 | % |
| UV386 | UV 386A | 0.171-0.189 | % |
| Water | | Balance | |

### Example 6 (AG76)

| | | Actual | |
|---|---|---|---|
| | Registry | Concentration | Units |
| PEGDA 3400 | 40% PEGDA 6000 | 7.6-8.4 | % |
| HEPES | 1M HEPES pH 7.3 | 0.095-0.105 | M |
| 10X PBS | DPBS 10X Ca, Mg —/— | 0.95-1.05 | X |
| HPA | Hydroxylpropyl Acrylate | 9.5-10.5 | % |
| | (HPA) | | |
| NAP | NAP | 1.425-1.575 | % |
| UV386 | UV 386A | 0.114-0.126 | % |
| Water | | Balance | |

### Print Settings for LD80 Printer

| Region 1 | Value | Units |
|---|---|---|
| Layer Thickness | 20 | μm |
| Irradiance | 13 | mW/Cm² |
| Cure Time | 1000 | ms |
| Pump Up Time | 1500 | ms |
| Pump Down Time | 1500 | ms |
| Pump Up Delay | 500 | ms |
| Pump Down Delay | 1500 | ms |
| Pump Speed | 300 | ms |
| Baselayer | | |
| Irradiance | 13 | mW/Cm² |
| Cure Time | 2000 | ms |
| Pump Up Time | 1000 | ms |
| Pump Down Time | 1000 | ms |
| Pump Up Delay | 1000 | ms |
| Pump Down Delay | 1000 | ms |
| Pump Speed | 1000 | ms |

FIG. 6 shows a photograph of exemplary 3D printed AM-transwells having a cylindrical shape using a synthetic bioink and a LS80 printer.

AM-transwells were reproducible using all synthetic bioink formulations and print settings in LS80 printer.

### Exemplary Bioink Ink Formulations and Print Settings for BLF20 Printer

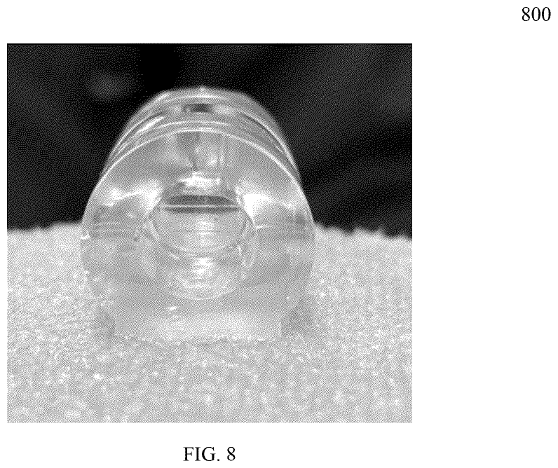

FIG. 7 depicts a photograph of an exemplary 3D printed AM-transwell having a cube shape using a synthetic bioink and a BLF20 printer; and FIG. 8 depicts a photograph of an exemplary 3D printed AM-transwell having a cylindrical shape using a synthetic bioink and a BLF20 printer.

### Example 7 (AG77)

| | | Actual | |
|---|---|---|---|
| | Registry | Concentration | Units |
| PEGTAC | PEGTAC | 2.85-3.15 | % |
| PEGDA 6000 | 40% PEGDA 6000 | 4.75-5.25 | % |
| HEPES | 1M HEPES pH 7.3 | 0.095-0.105 | M |
| 10X PBS | DPBS 10X Ca, Mg —/— | 0.95-1.05 | X |
| HPA | Hydroxylpropyl Acrylate | 9.5-10.5 | % |
| | (HPA) | | |
| NAP | NAP | 1.425-1.575 | % |
| UV386 | UV 386A | 0.152-0.168 | % |
| Water | | Balance | |

### Example 8 (AG78)

| | | Actual | |
|---|---|---|---|
| | Registry | Concentration | Units |
| PEGTAC | PEGTAC | 2.85-3.15 | % |
| PEGDA 6000 | 40 PEGDA 6000 | 4.75-5.25 | % |
| HEPES | 1M HEPES pH 7.3 | 0.095-0.105 | M |
| 10X PBS | DPBS 10X Ca, Mg —/— | 0.95-1.05 | X |
| | 29320006 | | |
| PHSRNK-RGDS | PHSRNKRGDS | 1.9-2.1 | mM |
| HPA | Hydroxypropyl Acrylate | 9.5-10.5 | % |
| | (HPA) | | |
| NAP | NAP | 1.425-1.575 | % |
| UV386 | UV 386A | 0.152-0.168 | % |
| Water | | Balance | |

### Example 9 (AG79)

| | | Actual | |
|---|---|---|---|
| | Registry | Concentration | Units |
| PEGDA 700 | PEGDA 700 | 3.325-3.675 | % |
| PEGDA 6000 | 40 PEGDA 6000 | 4.75-5.25 | % |
| HEPES | 1M HEPES pH 7.3 | 0.095-0.105 | M |
| 10X PBS | DPBS 10X Ca, Mg —/— | 0.95-1.05 | X |
| HPA | Hydroxypropyl Acrylate | 9.5-10.5 | % |
| | (HPA) | | |
| NAP | NAP | 01.425-1.575 | % |
| UV386 | UV 386A | 0.152-0.168 | % |
| Water | | Balance | |

### Example 10 (AG80)

| | | Actual | |
|---|---|---|---|
| | Registry | Concentration | Units |
| PEGDA 700 | PEGDA 700 | 3.325-3.675 | % |
| PEGDA 6000 | 40 PEGDA 6000 | 4.75-5.25 | % |
| HEPES | 1M HEPES pH 7.3 | 0.095-0.105 | M |
| 10X PBS | DPBS 10X Ca, Mg —/— | 0.95-1.05 | X |
| MMP | MMP—Crosslinker | 9.5-10.5 | mM |
| PHSRNK-RGDS | PHSRNKGDS | 1.9-2.1 | mM |
| HPA | Hydroxypropyl Acrylate | 9.5-10.5 | % |
| | (HPA) | | |
| NAP | NAP | 1.425-1.575 | % |
| UV386 | UV 386A | 0.152-0.168 | % |
| Water | | Balance | |

### Print Settings for BLF20 Printer (Cube Transwell Design)

| Region 1 | Value | Units |
|---|---|---|
| Layer Thickness | 50 | μm |
| Irradiance | 50 | mW/Cm² |
| Cure Time | 1000 | ms |
| Pump Up Time | 1500 | ms |
| Pump Down Time | 1500 | ms |
| Pump Up Delay | 500 | ms |
| Pump Down Delay | 1500 | ms |
| Pump Speed | 300 | ms |
| Baselayer | | |
| Irradiance | 50 | mW/Cm² |
| Cure Time | 2000 | ms |
| Pump Up Time | 1000 | ms |
| Pump Down Time | 1000 | ms |
| Pump Up Delay | 1000 | ms |
| Pump Down Delay | 1000 | ms |
| Pump Speed | 1000 | ms |

FIG. 7 depicts a photograph of an exemplary 3D printed AM-transwell having a cube shape using a synthetic bioink and a BLF20 printer.

### Print Settings for BLF20 Printer (Round (Cylindrical) Design)

| Region 1 | Value | Units |
|---|---|---|
| Irradiance | 13 | mW/Cm² |
| Cure Time | 4000 | ms |
| Step Speed | 150 | ms |
| Pump Every nth Layer | 1 | ms |
| Pump Distance | 300 | ms |
| Pump Up Time | 1500 | ms |
| Pump Up Time Delay | 500 | ms |
| Pump Down Time | 1500 | ms |
| Pump Down Time Delay | 1500 | ms |
| Speed to Build Start | 3335 | ms |
| Base Layer | | |
| Irradiance | 13 | mW/Cm² |
| Cure Time | 8000 | ms |
| Step Speed | 200 | ms |
| Pump Every nth Layer | 1 | ms |
| Pump Distance | 1000 | ms |
| Pump Up Time | 1000 | ms |
| Pump Time Delay | 1000 | ms |
| Pump Down Time | 1000 | ms |
| Pump Down Time Delay | 1000 | ms |
| Speed to Build Start | 3335 | ms |

FIG. 8 depicts a photograph of an exemplary 3D printed AM-transwell having a cylindrical shape using a synthetic bioink and a BLF20 printer.

AM-transwells were reproducible using all synthetic bioink formations and print setting in BLF19/20 and FS20 printers.

### Permeability of AM-Transwells

### AM-Transwell Printing

Printer vats were cleaned prior to loading bioink. After cleaning, 20 mL bioink was added to printer vat. Printer settings were loaded to digital light projection (DLP) printer and then printed. After printing, the 3D printed AM-transwells were offloaded into 1X PBS Ca+/Mn+. Printer vats and platforms were cleaned.

### AM-Transwell Sterilization

The 3D printed AM-transwells are offloaded into 1X PBS Ca+/Mn+. Scaffolds are washed three time in 1X PBS Ca+/Mn+. 1X PBS Ca+/Mn+ waste is disposed in an acylate waste container. The scaffolds and 3D printed AM-transwells are incubated overnight in 0.1 M HEPES/1 X PBS supplemented with antibiotic/antimycotic (anti-anti).

### Permeability Assay

Inside a biosafety cabinet, the sterilization buffer was decanted. After decanting, the 3D printed AM-transwells were transferred to a twelve well plate. 1.2 mL PAEC culture media was added to apical compartment (upper chamber). Any bubbles were gently removed from underneath the 3D printed AM-transwell.

Prepare FITC-dextran solution in biosafety cabinet with little to no light.

### Calculations for FITC-Dextran Solution

Final Experimental Concentration=400 μg/mL

Stock Concentration=10 mg/mL

Total Volume=15 mL

(400 μg/mL)(15 mL)/(10,000 μg/mL)=0.6 mL=600 μL FTIC-Dextran 10 K

300 μL of FITC-Dextran solution was added to apical compartment (upper chamber) of the 3D printed AM-transwells so that permeability into basal compartment (lower chamber) may be measure over time. 600 μL of FITC-Dextran solution was added to commercially available plastic transwells (e.g., Corning) as control.

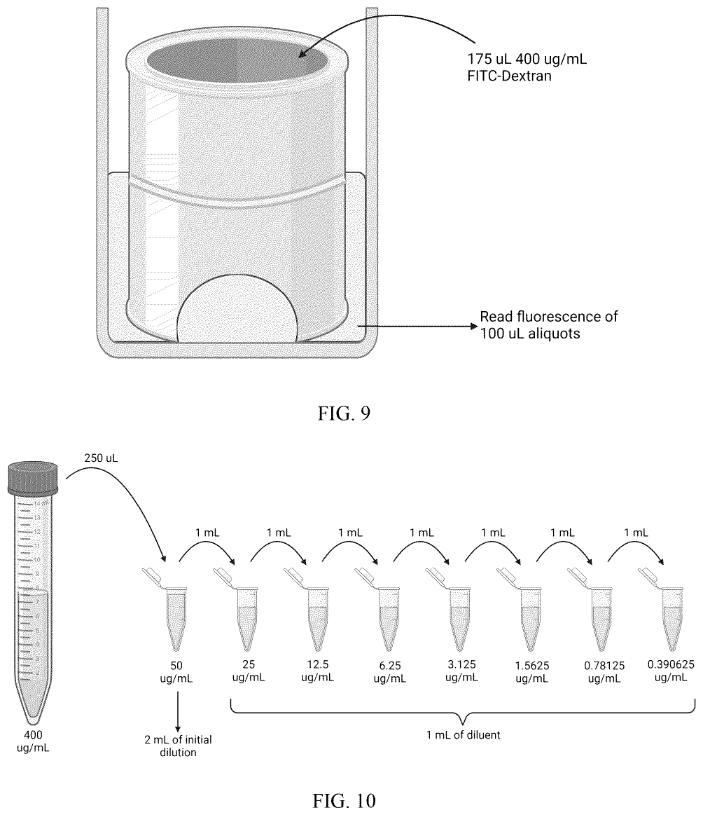

FIG. 9 shows a schematic of an experimental set-up for a permeability assay of AM-transwells.

Measure fluorescence of 100 μL aliquots (n=3) from basal chamber (lower compartment) at 1 hour, 2 hours and 4 hours at 490/520 nm on a plate reader.

Replenish FITC-Dextran media in basal compartment (lower chamber) in equal volume to the aliquots removed to measure fluorescence.

### Standard Curve Generation

FIG. 10 shows a schematic of an experimental set-up for a serial dilution established using known concentration of FITC-Dextran for the permeability assay of AM-transwells.

For standard curve, take 250 μL of the 400 μg/mL FITC-Dextran solution into 1.750 mL PAEC media. Pipette up and down to mix thoroughly. Transfer 1 mL of the dilution to an Eppendorf tube containing 1 mL PAEC media and mix thoroughly. This will dilute the original concentration in half. Continue for a total of eight dilutions. Measure fluorescence of 100 μL aliquots at 490/520 nm. Generate standard curve using known concentrations. Determine a best-fit line. Use best fit equation to quantify FITC-Dextran in experimental samples. In best-fit equation, x is concentration (μg/mL) and Y is relative fluorescence.

### Calculation for Standard Curve Initial Dilution

Initial Concentration=400 μg/mL

Final Concentration=50 μg/mL

(50 μg/mL)(2 mL)/(400 μg/mL)=0.25 mL

Diluent=2000-250=1750 μL

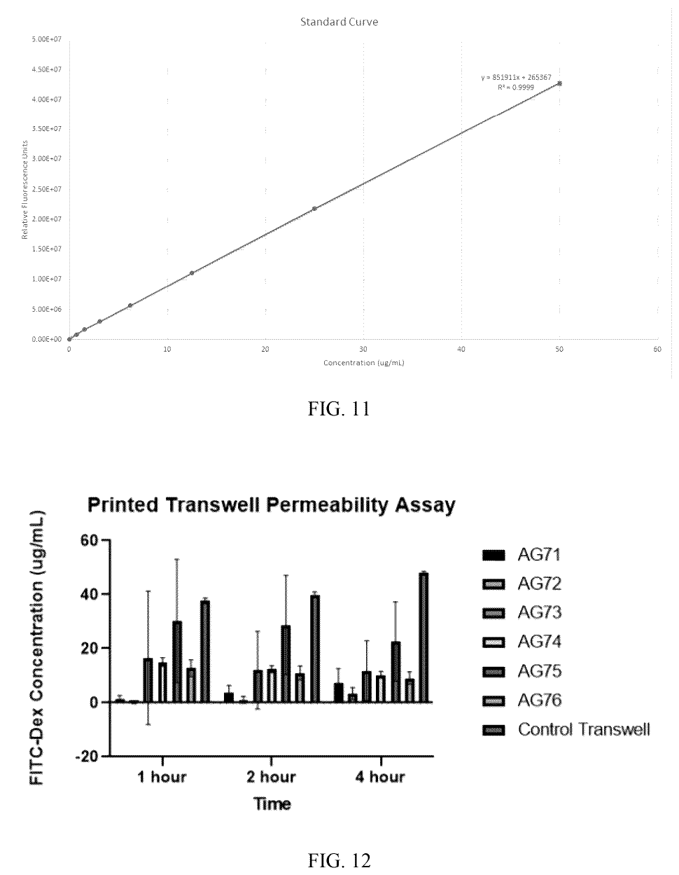

FIG. 11 shows a standard curve chart of Concentration (μg/mL) vs. Relative Fluorescence Units for the permeability assay of AM-transwells (AG71 through AG76 ).

FIG. 12 shows a chart of Time vs FITC-Dextran Concentration (μg/mL) for the permeability assay of AM-transwells (AG71 through AG76 ).

The concentration of FITC-Dextran in the 3D printed AM-transwells over time compared with the commercially available plastic transwells (e.g., Corning). A slight downward trend in concentration of the FITC-Dextran was observed for AG73 through AG76 while a slight upward trend in concentration of the FITC-Dextran was observed for AG71 and AG72.

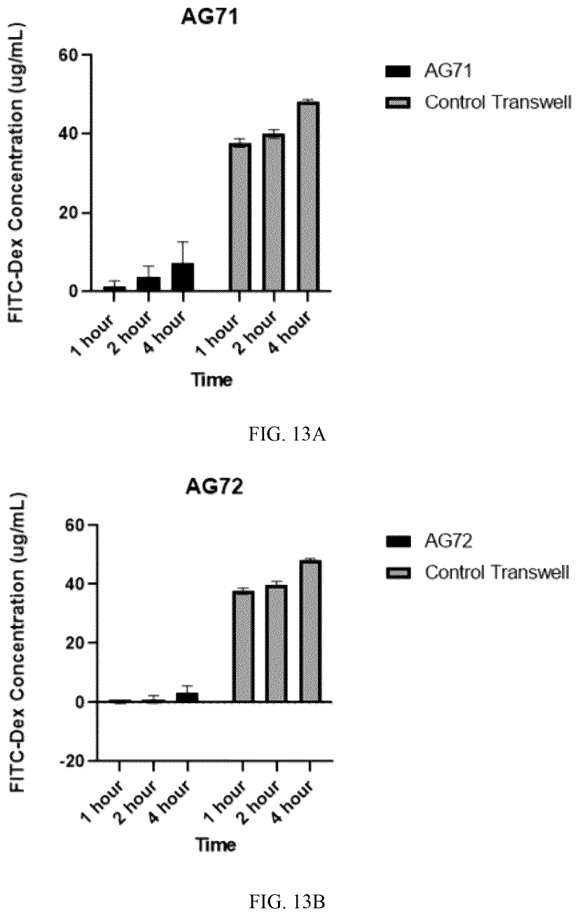

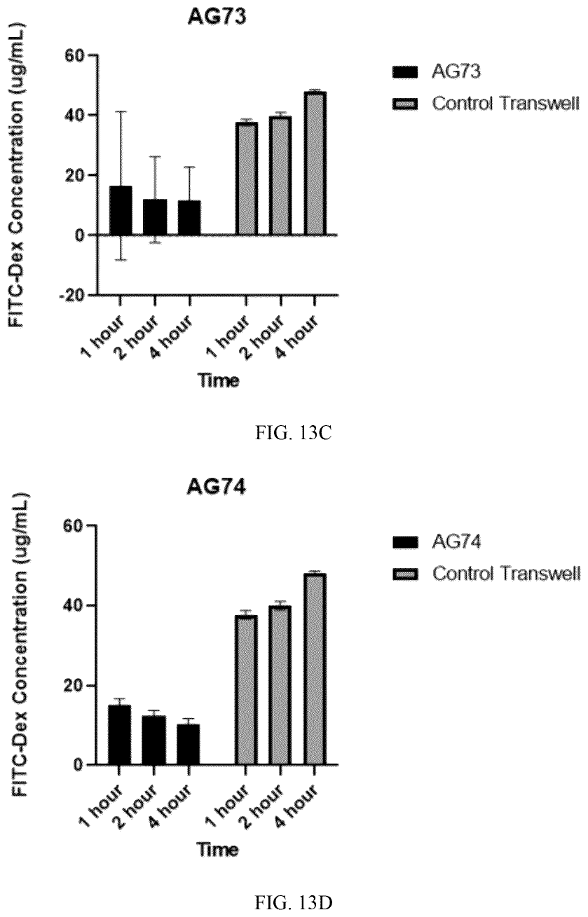

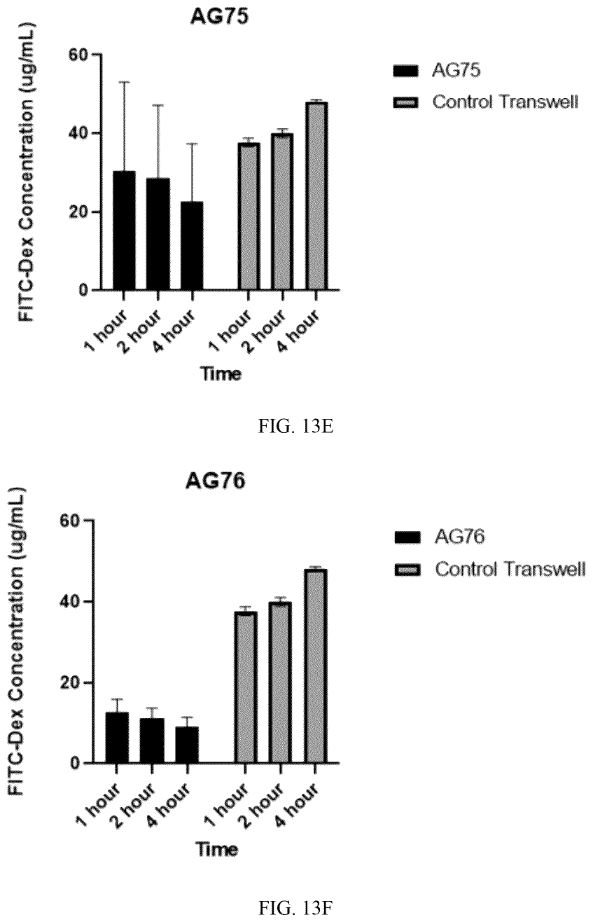

FIG. 13A shows a chart of Time vs. FITC-Dextran Concentration (μg/mL) for the permeability assay of an AM-transwells (AG71); FIG. 13B shows a chart of Time vs. FITC-Dextran Concentration (μg/mL) for the permeability assay of an AM-transwells (AG72); FIG. 13C shows a chart of Time vs. FITC-Dextran Concentration (μg/mL) for the permeability assay of an AM-transwells (AG73); FIG. 13D shows a chart of Time vs. FITC-Dextran Concentration (μg/mL) for the permeability assay of an AM-transwell (AG74); FIG. 13E shows a chart of Time vs. FITC-Dextran Concentration (μg/mL) for the permeability assay of an AM-transwell (AG75); and FIG. 13F shows a chart of Time vs. FITC-Dextran Concentration (μg/mL) for the permeability assay of an AM-transwell (AG76).

### Permeability Assay

Create a standard curve by preparing and measuring fluorescence of ½ serial dilutions starting at 50 μg/mL at 490/520 nm.

In a twelve-well plate, fill each well to be used with 1.2 mL PAEC culture media. Carefully, transfer 3D printed AM-transwells to twelve-well plate. Any bubbles were gently removed from underneath the 3D printed AM-transwell.

Once the bubbles are removed, add 175 μL of 400 μg/mL FITC-Dextran solution to apical compartment (upper chamber) of the 3D printed AM-transwell.

Cover the twelve-well plate in foil and incubate at 37° C.

FIG. 9 shows a schematic of an experimental set-up for a permeability assay of AM-transwells.

Measure fluorescence of 100 μL aliquots (n=3) from basal chamber (lower compartment) at 1 hour, 2 hours, 4 hours and 21 hours at 490/520 nm on a plate reader.

Replenish FITC-Dextran media in basal compartment (lower chamber) in equal volume to aliquots removed.

Use commercially available plastic transwells (e.g., Corning) as control. Standard Curve Generation

FIG. 10 shows a schematic of an experimental set-up for a serial dilution for the permeability assay of AM-transwells.

For the serial dilution take 250 μL of 400 μg/mL FITC-Dextran solution into 1.750 mL of endothelial (PAEC) media. Pipette up and down to mix thoroughly. Transfer 1 mL of this first dilution to a new Eppendorf tube containing 1 mL PAEC media and mix thoroughly. Continue for a total of eight sequential dilutions. Transfer 100 μL of each dilution (three replicates) into a 96-well plate and measure fluorescence at 490/520 nm. Generate standard curve using known concentrations. Determine a best-fit line. Use best fit equation to quantify FITC-Dextran in experimental samples. In best-fit equation, x is concentration (μg/mL) and Y is relative fluorescence.

### Calculations for FITC-Dextran Stocks

Stock=10 mg/mL

Final Assay Concentration=400 μg/mL

Final Volume=4 mL

Stock Volume=(400 μg/mL) (4 mL)/10,000 μg/mL)=0.16 mL=160 μL

### Calculation for Standard Curve Initial Dilution

Initial Concentration=400 μg/mL

Final Concentration=50 μg/mL

(50 μg/mL)(2 mL)/(400 μg/mL)=0.25 mL

Diluent=2000 μL-250 μL=1750 μL

### 21-Hour Analysis

After final 21-hour fluorescence measurement, remove FITC-Dextran media from apical compartment (upper chamber) of the 3D printed AM-transwells. Assuming the concentration is 400 μg/mL, dilute solution 1:16 for a theoretical concentration of 25 μg/μL. Take 100 100 μL aliquots and measure fluorescence of at 490/520 nm on plate reader.

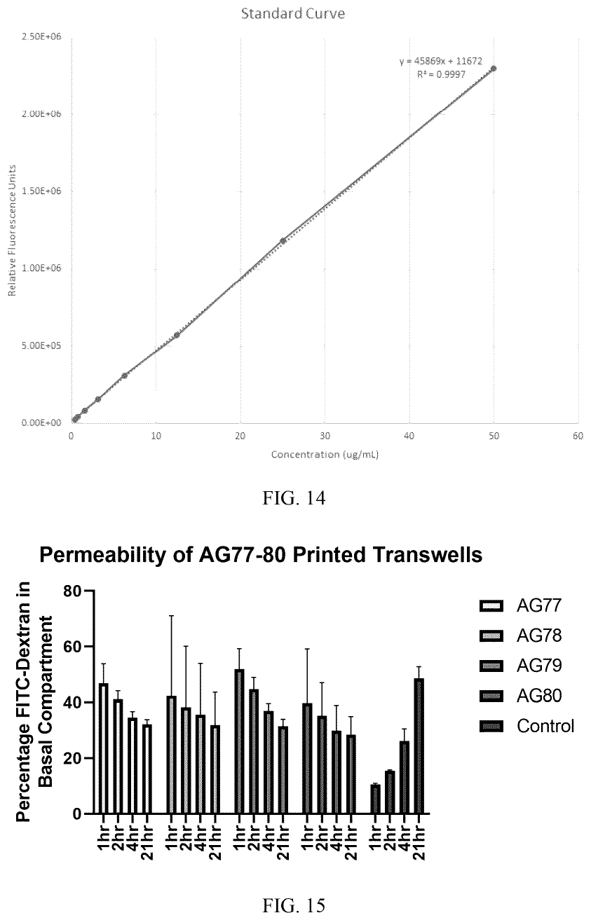

FIG. 14 shows a standard curve chart of Concentration (μg/mL) vs. Relative Fluorescence Units for the permeability assay of an AM-transwell (AG77 through AG80 ).

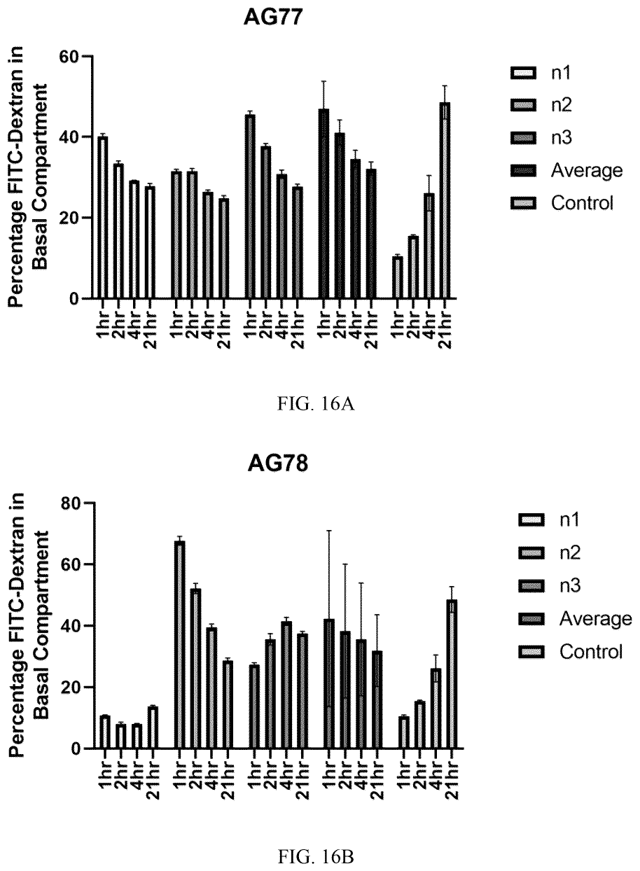

FIG. 15 shows a chart of Time vs. Percentage FITC-Dextran in Basal Compartment for the permeability assay of AM-transwells (AG77 through AG80 ).

The concentration of FITC-Dextran in the 3D printed AM-transwells over time compared with the commercially available plastic transwells (e.g., Corning). Data is represented as average of mass percent of FITC-Dextran in basal compartment (lower chamber) at time T compared to total FITC-Dextran in system.

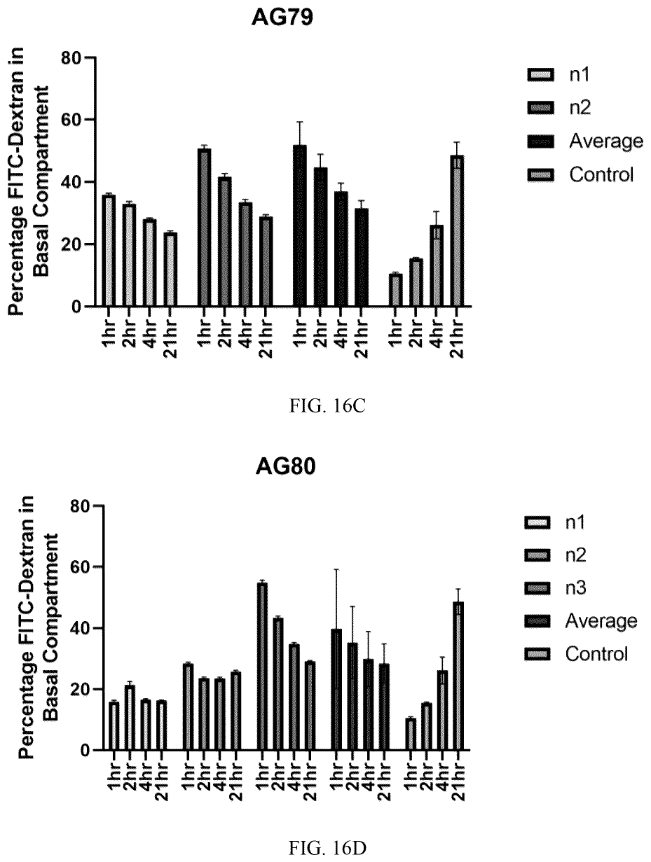

FIG. 16A shows a chart of Time vs. Percentage FITC-Dextran in Basal Compartment for the permeability assay of an AM-transwell (AG77); FIG. 16B shows a chart of Time vs. Percentage FITC-Dextran in Basal Compartment for the permeability assay of an AM-transwell (AG78); FIG. 16C shows a chart of Time vs. Percentage FITC-Dextran in Basal Compartment for the permeability assay of an AM-transwell (AG79); and FIG. 16D shows a chart of Time vs. Percentage FITC-Dextran in Basal Compartment for the permeability assay of an AM-transwell (AG80).

Permeability of each replicate, each fluorescence measurement measured in triplicate compared to average for the formulation and average of commercially available plastic transwell (e.g., Corning) as control. Data is represented as average of mass percent of FITC-Dextran in basal compartment (lower chamber) at time T compared to total FITC-Dextran in system.

### Media Recovery After Three Days

### Method

Liquid volume changes in apical compartment (upper chamber) and basal compartment (lower compartment) of 3D printed AM-transwell (AG71) after a period of time was determined. The 3D printed AM-transwell were first stored in PBS in 50 mL tubes. The PBS was decanted and the 3D printed AM-transwells transferred to sterile 50 mL tubes. 35 mL HEPES/PBS anti-anti was added to 50 mL tubes with 3D printed AM-transwells and sterilized overnight. After sterilization, the sterilization buffer was decanted, and the 3D printed AM-transwells were incubated in media for at least 4 hours. After media incubation, the 3D printed AM-transwells were transferred to a twelve-well plate. 1.2 mL media was added to the basal compartment of the 3D printed AM-transwell. Any bubbles were gently removed from underneath the 3D printed AM-transwell. After the bubble were removed, 0.5 mL media was carefully added to a basal compartment of the 3D AM-transwell. 175 μL media was added dropwise to apical compartment carefully to avoid any overflow. The 3D printed AM-transwells were incubated for three days at 37° C. in an incubator. After the third day, the plate was removed from the incubator. For each 3D printed AM-transwell, all of the media was recovered from the basal compartment into a 5 mL tube. Similarly, all of the media was carefully recovered from the apical compartment into a 0.5 mL Eppendorf tube. The total volume of media recovered from each compartment was then measured and recorded.

### Initial Conditions

| Formulation | AG71 | Units |
|---|---|---|
| Number of AM-Transwells | 3 | Each |
| Initial Apical Volume | 175 | μL |
| Initial Basal Volume | 1.7 | mL |

### Apical Recovery

| | Final Apical | | Percentage | |
|---|---|---|---|---|
| Number | Volume | Units | Recovered | Units |
| N1 | 60 | μL | 34.3 | % |
| N2 | 70 | μL | 40.0 | % |
| N3 | 76 | μL | 43.4 | % |

Basal Recovery

| | Final Basal | | Percentage | |
|---|---|---|---|---|
| Number | Volume | Units | Recovered | Units |
| N1 | 1.68 | mL | 98.8 | % |
| N2 | 1.78 | mL | 104.7 | % |
| N3 | 1.65 | mL | 97.1 | % |

### Total Recovery

| | Final Total | | Percentage | |
|---|---|---|---|---|
| Number | Volume | Units | Recovered | Units |
| N1 | 1.74 | mL | 92.8 | % |
| N2 | 1.85 | mL | 98.7 | % |
| N3 | 1.73 | mL | 92.1 | % |

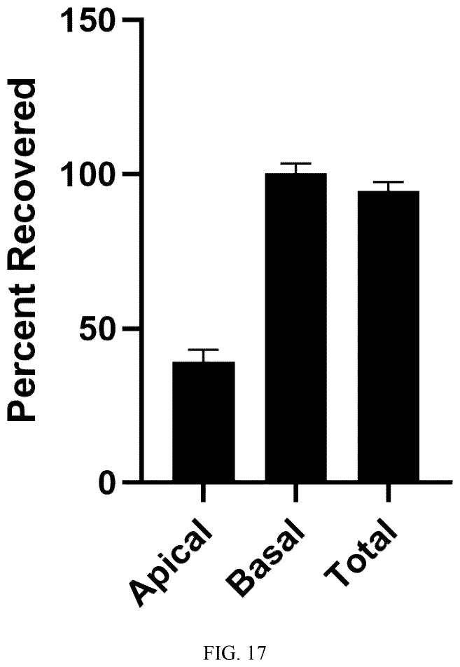

FIG. 17 shows a chart of Compartment vs. Percent Media Recovered from an AM-transwell after three days.

Volume of apical compartment (upper chamber) could be increased to hold more media.

More media could be added to the outside of the AM-transwell to allow the hydrogel to stay more hydrated and to retain some media in the apical compartment.

Media could be changed daily when culturing cells to prevent the apical compartment from becoming too dry to support cell monolayers as indicated by the >50 % remaining media in the apical compartment after three days.

### Method of Making AM-Transwell

A method of making the AM-transwell comprises: a) printing one or more of a lower chamber, an upper chamber and a membrane of the AM-transwell using a 3D printing technique; b) assembling and/or printing the AM-transwell as described herein to form assembled AM-transwells.

In an embodiment, step b) prints at least 3 assembled AM-transwells. In an embodiment, step b) prints at least 20 assembled AM-transwells. In an embodiment, step b) prints at least 50 assembled AM-transwells.

In an embodiment, the method further comprises: c) optionally, storing the assembled AM-transwells at 4° C. until needed.

In an embodiment, the method further comprises: c) transferring each of the AM-transwells into DPBS+/+ in tubes; d) decanting the DPBS+/+ from the tubes and washing each the AM-transwells two additional times in DPBS+/+ for about 5 minutes to form first washed AM-transwells; and e) transferring the first washed AM-transwells to sterile 50 mL tubes.

In an embodiment, the method further comprises: f) incubating the first washed AM-transwells in DPBS−/− 1X anti-anti overnight to form incubated AM-transwells.

In an embodiment, the method further comprises: g) decanting the DPBS−/− 1X anti-anti and washing the incubated AM-transwells in about 35 PBS/HEPES 1X anti-anti for about 4 hours to form second washed AM-transwells; and h) decanting the PBS/HEPES 1X anti-anti from the tubes and incubating the second washed AM-transwells in about 35 mL cell culture media in about 50 mL tubes for about 2 days or over a weekend.

In an embodiment, the 3D printing technique is one or more of digital light projection printing (DLP), stereolithography (SLA) printing technique, extrusion 3D printing technique or selective laser sintering 3D printing technique or a combination thereof. In an embodiment, the 3D printing technique is a digital light printing (DLP) printing technique.

### Method of Using AM-Transwell

A method of using the AM-transwell comprises: a) cell seeding at least one side of the membrane of the AM-transwell as described herein.

### Method of Using AM-Transwell

A method of using the AM-transwell comprises: a) cell seeding both sides of the membrane of the AM-transwell as described herein for in vitro cells studies.

The embodiments and examples set forth herein are presented to best explain the present invention and its practical application and to thereby enable those skilled in the art to make and utilize the invention. However, those skilled in the art will recognize that the foregoing description and examples have been presented for the purpose of illustration and example only. The description as set forth is not intended to be exhaustive or to limit the invention to the precise form disclosed. Many modifications and variations are possible in light of the above teaching without departing from the spirit and scope of the following claims. The invention is specifically intended to be as broad as the claims below and their equivalents.

Any references to implementations or elements or acts of the systems and methods herein referred to in the singular can include implementations including a plurality of these elements, and any references in plural to any implementation or element or act herein can include implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

Any implementation disclosed herein may be combined with any other implementation, and references to “an implementation,” “some implementations,” “an alternate implementation,” “various implementations,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Elements other than ‘A’ and ‘B’ can also be included.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

1.-29. (canceled)

30. An advanced manufactured transwell (AM-transwell), the AM-transwell comprising:
a) a lower chamber;
b) an upper chamber;
c) a membrane disposed between the lower chamber and the upper chamber; and
d) one or more legs, wherein the one or more legs form at least a portion of the lower chamber, wherein one or more of the lower chamber, the upper chamber, the membrane and the one or more legs is printed using a synthetic bioink.

31. The AM-transwell of claim 30, wherein the AM-transwell is a cylinder, a cube, a cuboid, a truncated cone, a truncated pyramid, a truncated sphere or a combination thereof.

32. The AM-transwell of claim 30, wherein the AM-transwell is a cylinder.

33. The AM-transwell of claim 30, wherein the AM-transwell has a height of about 10 mm to about 16 mm and a thickness from about 1 mm to about 3 mm.

34. The AM-transwell of claim 30, wherein the AM-transwell has a height of about 13 mm and a thickness of about 2 mm.

35. The AM-transwell of claim 30, wherein the lower chamber has a height from about 2 mm to about 4 mm and the upper chamber has a height from about 8 to about 12 mm.

36. The AM-transwell of claim 30, wherein the lower chamber has a height of about 3 mm and the upper chamber has a height of about 10 mm.

37. The AM-transwell of claim 30, wherein the membrane has a thickness from about 1 mm to about 3 mm.

38. The AM-transwell of claim 30, wherein the membrane has a thickness of about 2 mm.

39. The AM-transwell of claim 30, wherein the synthetic bioink comprises one or more of a degradable peptide ink and triacrylate peptide ink.

40. The AM-transwell of claim 30, wherein the synthetic bioink comprises one or more of:
HPA, in an amount from about 3% to about 10%;
PEGDA3400, in an amount from about 5% to about 20%;
PEGDA6000, in an amount from about 5% to about 20%;
PEGDA575, in an amount from about 1% to about 20%;
PEGDA700, in an amount from about 1% to about 20%;
PEGTAC, in an amount from about 1% to about 5%;
PEO, in an amount from about 0.1% to about 5%;
NAP, in an amount from about 1% to about 3%;
LAP, in an amount from about 1% to about 3%; and
UV386 A, in an amount from about 0.1% to about 0.5%.

41. The AM-transwell of claim 40, wherein the synthetic bioink further comprises:
water, in an amount as a balance.

42. The AM-transwell of claim 40, wherein the synthetic bioink further comprises:
a buffer solution comprising 0.1 M HEPES in water and 1X PBS at pH of 7.2.

43. The AM-transwell of claim 40, wherein the synthetic bioink further comprises:
mono-cysteine peptide, in an amount from 0.5 mM to 20 mM; and
dicysteine peptide, in an amount from 0.5 mM to 20 mM.

44. The AM-transwell of claim 43, wherein the mono-cysteine peptide comprises one or more of RGDS (SEQ ID NO: 1 ), PHSRNKRGDS (SEQ ID NO: 2 ), IKVAV (SEQ ID NO: 3 ), Bm-binder, and FN-binder.

45. The AM-transwell of claim 43 wherein the membrane is seeded with cells.

46. A method of making an advanced manufactured transwell (AM-transwell) comprising:
a) printing one or more of a lower chamber, an upper chamber and a membrane of the AM-transwell using a 3D printing technique; and
b) assembling and/or printing the AM-transwell of claim 1 to form assembled AM-transwells.

47. The method of claim 46, wherein the 3D printing technique is one or more of digital light projection printing (DLP), sterolithography (SLA) printing technique, extrusion 3D printing technique or selective laser sintering 3D printing technique or a combination thereof.

48. A method of using an advanced manufactured transwell (AM-transwell) comprising cell-seeding both sides of a membrane of the AM-transwell of claim 1 for an in vitro cell study.


SEQUENCE LISTING (5 sequences)

SEQ ID NO: 1 - protein, 4 aa, synthetic construct
  source 1..4: note = mono-cysteine peptide
RGDS

SEQ ID NO: 2 - protein, 10 aa, synthetic construct
  source 1..10: note = mono-cysteine peptide
PHSRNKRGDS

SEQ ID NO: 3 - protein, 5 aa, synthetic construct
  source 1..5: note = Seq ID_3 mono-cysteine peptide
IKVAV

SEQ ID NO: 4 - protein, 6 aa, synthetic construct
  source 1..6: note = mono-cysteine peptide
GFOGER

SEQ ID NO: 5 - protein, 12 aa, synthetic construct
  source 1..12: note = mono-cysteine peptide_AG73
RKRLQVQLSIRT